(19) United States
(12) Patent Application Publication
(10) Pub. No.: US 2003/0111084 A1
(43) Pub. Date: Jun. 19, 2003

(54) Use of low-power laser irradiation for enhanced vascularization of tissue and tissue-engineered construct transplants

(76) Inventors: Elena Revazova, Los Angeles, CA (US); Jeff Sebastian, Los Angeles, CA (US)

Correspondence Address: Laurie A. Axford, Morrison & Foerster LLP, Suite 500, 3811 Valley Centre Drive, San Diego, CA, 92130, US

(21) Appl. No.: 10/233,004

(22) Filed: Aug. 30, 2002

Related U.S. Application Data
(60) Provisional application No. 60/316,350, filed on Aug. 30, 2001.

(51) Int. Cl. A61N 1/00; A61B 19/00

(52) U.S. Cl. 128/898; 607/89

(57) ABSTRACT

The success of tissue transplantation, when immunological conflict is minimized, depends on the vascularization process. This process is very complex and requires time. During this time, transplanted tissue often has difficulty obtaining oxygen and nutrients. These factors have a profound influence on the survival of the transplanted tissue, especially if the tissue has poor angiogenic properties. For new vessel formation, endothelial cells from existing recipient microvessels must proliferate and migrate through the extracellular matrix into the transplanted tissue. However, if transplanted tissue is irradiated with low-power laser, vascularization and acceptance of auto-, allo-, and heterotransplants is enhanced.

This application claims priority under 35 U.S.C. 119(e) to provisional application No. 60/316,350 filed Aug. 30, 2001, the contents of which are incorporated herein by reference.

### FIELD OF THE INVENTION

This invention relates to the vascularization of transplants. More particularly, it relates to the use of low-power irradiation to enhance vascularization of tissue and tissue-engineered construct transplants.

### BACKGROUND OF THE INVENTION

Transplantation of organs, tissues, and tissue-engineered constructs is now commonplace in the treatment of a variety of medical conditions. For example, organs such as hearts and kidneys are now routinely transplanted in order to replace diseased organs. In addition, bone marrow transplantation is commonly performed as a treatment for leukemia and other hematological diseases. Other tissues, such as skin, are transplanted for a variety of reasons. More recently, complex tissue-engineered constructs have been prepared from biological and synthetic matrices containing various growth factors, therapeutics and/or cells. Collectively, these types of treatments can be referred to as “tissue transplantation”.

Solid organ transplants, such as heart, liver and kidney, are revascularized immediately upon reperfusion after the transplantation procedure. In contrast, the introduction of tissues such as islet cells, as well as various 2- and 3-dimensional tissue-engineered constructs (i.e. non-organ transplants) depends on new vessel formation, or “angiogenesis”, to ensure that the transplanted tissue receives an adequate blood supply. Accordingly, the success of tissue transplantation, when immunological conflict is absent (e.g. with auto- and singenic-transplantation, or allo- and heterotransplantation with immunodepletion), is highly dependent on the angiogenesis process (J. Folkman and M. Klagsbrun, Science 235:442-447 (1987); and C. H. Blood and B. R. Zetter, Biochim. Biophys. Acta, 1032(1): 89-118 (1990). This process is very complex and requires a significant amount of time, especially when the angiogenic properties of the transplanted tissue are compromised. It is during this time when the transplant is very vulnerable, because of a tenuous oxygen and nutrient supply, particularly in the more central portions of the transplant which are less reachable by diffusion. Thus, the post-transplantation angiogenic process has a profound influence on the survival of transplants such as islets, particularly when vascularization of pancreatic islet transplants can take longer than 7 days (A. M. Davalli et al., Diabetes 19:1161-1167 (1996)). In addition, the effects of hypoxia on the survival of islet cells has been studied (K. E. Dionne, et al., Diabetes 42:12-21 (1993)).

Various metabolic processes may also influence transplant survival. Because of this, transplantation of isolated pancreatic islets as a treatment modality for diabetes mellitus has had limited success (J. 1. Stranger, et al., Transplantation Proceedings 27(6): 3251-3254 (1995)). It is well documented that insulin secretion from transplanted islets is delayed and diminished when compared with secretion from a normal or transplanted pancreas. It has been suggested that a primary reason for nonimmune islet transplantation failure and inadequate insulin secretion may be the result of angiogenic inefficiency. Because transplanted islets require approximately 7 to 30 or more days for revascularization, it was suggested that this prolonged period of ischemia may be responsible for inadequate long-term beta-cell performance. This is quite understandable, since the beta-cells are located in the central portion of the islet where revascularization would take place last.

Angiogenesis is also an extremely complex process. It begins with the local dissolution of the basement membrane of an existing microvessel under the influence of endothelial derived proteases (D. Moscatelli and D. B. Rifkin, Biochem. Biophys. Acta., 948:67-85 (1988); and R. Montesano, et. al, Cell, 62: 435-445 (1990)). This is followed by endothelial cell proliferation and migration through the extracellular matrix toward the angiogenic stimulus (J. Folkman and M. Klagsbrun, Science 235:442-447 (1997); and J. Bauer, et al., J. Cellular Physiol. 153:437-449 (1992)). Finally, there is alignment of the migrating cells and formation of tubular structures. Microvascular tubes anastamose, forming a new capillary network through which blood flow is established.

Angiogenesis is also dependent on a complex signaling process that consists of two sets of extracellular signals. First, there are soluble factors that influence endothelial cell growth and differentiation. A very important group of soluble factors includes the heparin binding molecules that are related to acidic and basic fibroblasts growth factors (FGFs), as well as endothelial cell growth factor (ECGF) (W. H. Burgess and T. Maciag, Annual Rev. Biochem. 58: 575-606 (1989)). Other soluble factors that affect angiogenesis include TGF-beta, which inhibits proliferation and enhances differentiation of endothelial cells in vitro (M. S. Pepper, et al., J. Cell Biol. 111: 743-755 (1990)); platelet-derived growth factor (PDGF), which is among the most potent stimuli for cell migration in many cell types (J. Yu, et al., Biochem. Biophys. Res. Comm., 282(3): 697-700 (2001)); hypoxia-inducible factor 1 alpha (HIF-1 alpha) (E. Laughner, et al., Molecular and Cellular Biology 21(12): 3995-4004 (2001)); IL-1 and TNF (J. A. M. Maier, et al., Science 249: 1570-1574 (1990)); angiogenin, certain prostaglandins, and other low molecular weight substances (J. Bauer, et al., J. Cellular Physiol. 153: 437-449 (1992)).

The second major set of signals that regulate angiogenesis come from the extracellular matrix (M. Klagsbrun, J. Cell. Biochem. 47: 199-200 (1991)). Endothelial cell surface receptors of the integrin superfamily recognize extracellular matrix proteins that trigger a signaling event (S. M. Albelda C. A. Buck, FASEB J. 4 2868-2880 (1990)). It is suggested that the role of integrins is to maintain adhesive contact with the matrix and thus permit cell locomotion. However, this interaction may actually be more complex (J. Bauer, et al., J. Cellular Physiol. 153: 437-449 (1992)).

There have been attempts to improve the vascularization of transplanted pancreatic islets using acidic fibroblast growth factor. When syngeneic rat pancreas islets were transplanted into a kidney in the presence of this growth factor, the result was that more capillaries served the beta-cell-containing islet medulla, and a greater number of beta cells produced insulin (J. I. Stranger, et al., Transplantation Proceedings 27(6): 3251-3254 (1995)).

It has been previously demonstrated that low-power laser irradiation stimulates the proliferation and motility of cells. See, for example, M. Boulton and J. Marshall, Lasers in the Life Sciences, 1(2):125-134 (1986); P. Noble, et al., Lasers in Surgery and Medicine, 12:669 674 (1992); E. Glassberg, et al., Lasers in Surgery and Medicine, 8:567-572 (1988); and W. Yu, et al., Photochemistry and Photobiology, 59(2):167-170 (1994). The present invention relates to the use of low-power laser irradiation to enhance vascularization of organ, tissue and tissue-engineered construct transplants.

### SUMMARY OF THE INVENTION

The invention provides an improved method for transplantation which enhances vascularization. This is achieved by applying low power laser radiation to the transplanted tissue.

Thus, in one aspect, the invention is directed to a method of transplanting tissue into a recipient so as to enhance vascularization of said tissue which method comprises implanting a tissue prepared for transplant into a transplantation site in the recipient in applying low power laser to the tissue.

In one embodiment, the transplanted tissue is allogenic islets.

### BRIEF DESCRIPTION OF THE DRAWINGS

FIG. 1 shows the rabbit renal transplant (magnification 200×, stained with hematoxylin-eosin) after irradiation (A) and without irradiation (B)

FIG. 2 shows the rabbit pancreas transplant (magnification 200×, stained with hematoxylin-eosin) after irradiation (A) and without irradiation (B).

### DETAILED DESCRIPTION OF THE INVENTION

The present invention relates to the enhanced vascularization of transplants using low-power irradiation.

The Transplant

The present invention relates to tissue transplants as well as organ transplants. Tissue transplants may include, inter alia, bone, skin, connective tissue, heart tissue (including heart valves), vascular tissue and corneas. Organ transplants, on the other hand, include transplantation of whole organs such as the liver, kidneys, heart, lungs and pancreas. Unlike organ transplants that are performed less often in a fewer number of selected hospitals, tissue transplants are performed routinely at the majority of hospitals. In addition, there are important differences between the recovery of organs and tissues. Organs are recovered intact soon after death and require no processing before use. Tissue, on the other hand, can be recovered up to 24 hours after death and can be preserved through processes like freeze-drying and cryopreservation.

In the practice of the present invention, the transplant may comprise whole organs or organ fragments, tissue that is of natural origin, such as skin or bone marrow, or it may be cultured for purposes of transplantation. In addition, the transplant may comprise tissue-engineered constructs that are composed generally of a biological or synthetic matrix containing cells, which may also include various therapeutic agents and growth factors.

Skin transplants, more often called grafts, usually involve transplantation of a subject's own skin from one part of the body to another part of the body where the skin has sustained damage to the regenerative layers. This is called an “autograft”. Recent advances in cell culture techniques have contributed further to the success of skin transplants. It is now possible to remove a small section of skin from a burn victim, and grow it under controlled laboratory conditions. From an initial small sample, large sheets of epidermis have been grown and used to cover burn areas.

In contrast to autografts, “allografts” are transplants between individuals of the same species, and “heterogenic transplants” are transplants from different species. These transplants are complicated because of immunologic differences between donors and recipients that may result in rejection. However, the risk of rejection can be minimized by using various techniques to select donor tissues with enhanced compatibility, as well as the use of immunosuppressants, such as cyclosporine, to minimize the effects of the immune response following transplantation. However, the use of the immunosuppressants must be balanced against the risk of allowing the recipient to be vulnerable to pathogens, which could take full advantage of a compromised immune system.

Synthetic tissue-engineered constructs are also now used to transplant cells and tissues to treat a variety of different medical conditions. Tissue engineering involves the development of synthetic materials or devices that are capable of specific interactions with biological tissues. The constructs combine these materials with living cells to yield functional tissue equivalents. Such systems are useful for tissue replacement where there is a limited availability of donor organs or where, in some cases, (e.g., nerves) natural replacements are not readily. As used herein, the term “tissue-engineered constructs” includes any combination of naturally derived or synthetically grown tissue or cells, along with a natural or synthetic scaffold that provides structural integrity to the construct.

Tissue engineering involves a number of different disciplines, such as biomaterial engineering, drug delivery, recombinant DNA techniques, biodegradable polymers, bioreactors, stem cell isolation, cell encapsulation and immobilization, and the production of 2 dimensional and 3 dimensional scaffolds for cells.

Porous biodegradable biomaterial scaffolds are required for the 3 dimensional growth of cells to form the tissue engineering constructs. There are several techniques to obtain porosity for the scaffolds. Of these methods, fiber bonding, solvent casting/particulate leaching, gas foaming/particulate leaching and liquid-liquid phase separation produce large, interconnected pores to facilitate cell seeding and migration. The fiber bonding, solvent casting/particulate leaching and gas foaming/particulate leaching methods exhibit good biocompatibility, making these techniques especially promising for future use in tissue-engineered cell-polymer constructs.

Generally, the pores must be a size range that permits infiltration of a variety of different cells to grow within the scaffolds. In addition, depending on the size and shape of the construct, the scaffold must be biodegradable or porous enough to permit infiltration of endothelial cells and eventual angiogenesis.

In one aspect of the present invention, the tissue transplant is a pancreatic islet that is transplanted for the purpose of treating diabetes. There are two types of diabetes. Type I, which is the early onset form of diabetes, is characterized by immune-mediated destruction of the pancreatic islets. Patients with Type I diabetes become dependent on insulin for survival. In contrast, Type II diabetes is characterized by insulin resistance due to a lack of effective interaction between insulin and target cells. This type of diabetes usually occurs later in life and may or may not require insulin therapy.

There are many problems associated with long-term diabetes, despite use of insulin, such as blindness, renal failure, neuropathy and arteriosclerosis. Although many advances have been made to more closely mimic normal insulin production, these problems arise from the inability to tightly balance blood glucose concentrations. Accordingly, many physicians have turned to pancreatic organ transplant, which is usually performed together with a kidney transplant, to attempt to preserve normal body functions of Type I diabetes patients. However, in many patients, the risk of such an invasive treatment is often prohibitive.

More recently, researchers have developed various means of isolating and transplanting the islets of Langerhans (“islets”) from the pancreas separately. Islets are made up of two types of cells: the alpha cells, which make glucagon, a hormone that raises the level of glucose (sugar) in the blood, and the beta cells, which make insulin. Within the human pancreas organ there are about 1-1.5 million islets of Langerhans. The islets make up about 2% of the mass of the pancreas, and Each islet contains between 2,000 and 10,000 cells.

In addition to transplantation of in-tact islets, many attempts have been made to transplant tissue-engineered constructs containing beta-cells. However, just as with in-tact islets, it is important to promote microvascularization of the construct to enable the insulin secreted from the beta-cells to enter the general circulation, and also to provide the beta-cells with a source of oxygen and other nutrients.

The Laser-Irradiation

Surgical lasers such as carbon dioxide, helium-neon, argon and Nd:Yag lasers are widely used in a variety of different medical procedures. In general, they are capable of focusing laser light onto a precise target area. High-power laser primarily function by causing localized thermal effects, such as protein denaturation and vaporization. In contrast, low-power laser causes nonthermal effects on the target tissue, such as metabolic changes. Such effects have been referred to as “laser biostimulation” (W. Yu, et al., Photochemistry and Photobiology, 59(2):167-170 (1994)). Biostimulation is thought to occur between fluences of 0.05 and 10 J/cm² and emission power of approximately 1 to 15 mW (i.e. “low-power irradiation”), whereas the effects of higher intensities can actually inhibit metabolism.

Low-power laser administration is usually performed with red (630 nm) or near infrared (830 nm) laser light. Typical accumulated doses per area are of the order of 20 or less Joules per square centimeter.

The laser can be applied to either the transplant (before or after implantation) or the site of transplantation or both. Preferably, the laser is applied to the transplant after implantation at predetermined intervals. For example, the laser can be administered at days 1, 3, 5, etc., until an optimal amount of laser has been administered. In addition, the laser can be applied in a continuous manner or it can be pulsed

Laser equipment for medical uses is readily commercially available. For example, Softlaser 632 (World Laser Industries) is a He/Ne laser that can be set to emit an energy density of 1.5 j/cm². The Candela Vbeam is a pulsed dye 595 nm laser with variable parameters to treat common vascular lesions, scars and conditions like rosacea.

Transplantation with Recipient Endothelial Cells

In one aspect of the present invention, vascularization of the transplant is further enhanced using recipient endothelial cells. As used herein, the phrase “transplanting tissue” refers to both the transplantation of tissue from culture or from natural sources, as well as transplantation of tissue-engineered constructs that include tissue or cells. The transplant can be pretreated with recipient endothelial cells immediately prior to transplantation, transplanted simultaneously with the endothelial cells or, especially with tissue-engineered constructs, the transplant can be cultured with endothelial cells to enhance infiltration into the transplant prior to transplantation.

In most cases, it is sufficient to pretreat the transplant immediately before transplantation or to simultaneously administer endothelial cells to the site of transplantation. In any event, it is necessary to administer the endothelial cells in such a manner that they enhance vascularization of the transplant (i.e. it occurs faster and to a greater extent than if the recipient's endogenous endothelial cells were permitted to infuse the transplant.) However, it should be pointed out that the present invention does not intend transplantation of preformed vascular beds or other vascular structures, either separately or within the transplant, which would be time consuming and impractical using recipient endothelial cells.

Optional Embodiments

Various optional constituents, such as immunosuppressive agents, growth factors and other substances, can also be included with the endothelial cells and/or the transplant. Such constituents include, inter alia, extracellular matrix proteins such as collagen and fibronectin; integrins; growth factors such as tissue growth factors, etc. In particular, angiogenic factors can be administered along with the transplant, which include basic fibroblast growth factor, acidic fibroblast growth factor, endothelial cell growth factor, angiogenin, and transforming growth factors alpha and beta. Other optional transplant constituents are discussed in the background of invention.

### EXAMPLE

Renal tissue or pancreatic tissue from two-day old rabbits was quickly and thoroughly minced with a pair of sharp curved scissors under sterile conditions. The mince was then suspended in MEM 1:1, and 0.5 ml was injected subcutaneously into six-week old nude BALB/c mice. Injection sites were irradiated with He/Ne laser ODER ˜633 nm, 3.5 J/cm² at 1,3,5,8,10 and 12 days following transplantation. Six hour after the last irradiation, the transplants were removed and fixed with 10% neutral formaldehyde, and serial sections (7 millimicron thickness) were prepared and stained with hematoxylin-eosin. The number of blood vessels in the transplants was estimated by determining the mean number of vessels per section. Vessels were counted in 10 fields of vision in every fifth section at 250× magnification.

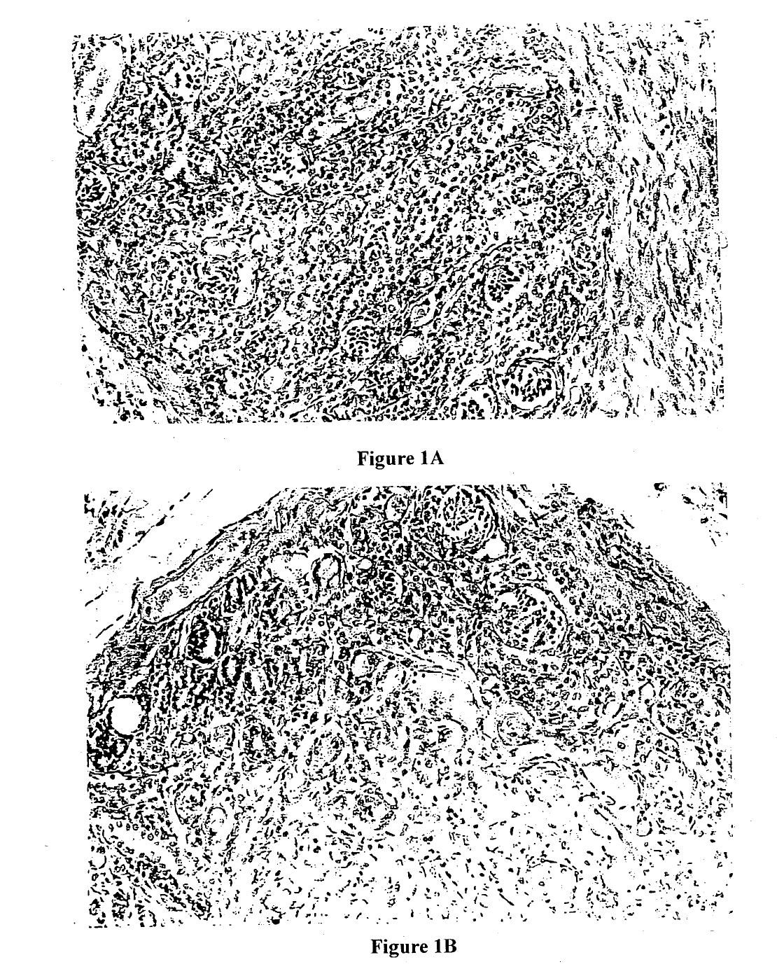

The mean transplant size and number of vessels were determined 33 days following injection. Blood vessels in the irradiation group were larger than vessels in the control group. In addition, the renal transplant group receiving irradiation exhibited structures typical of the renal cortex (glomeruli, winding and straight tubules, collecting tubules, etc.) as shown in FIG. 1A. In the non-irradiated group, the organ-specific structure was not observed as shown in FIG. 1B.

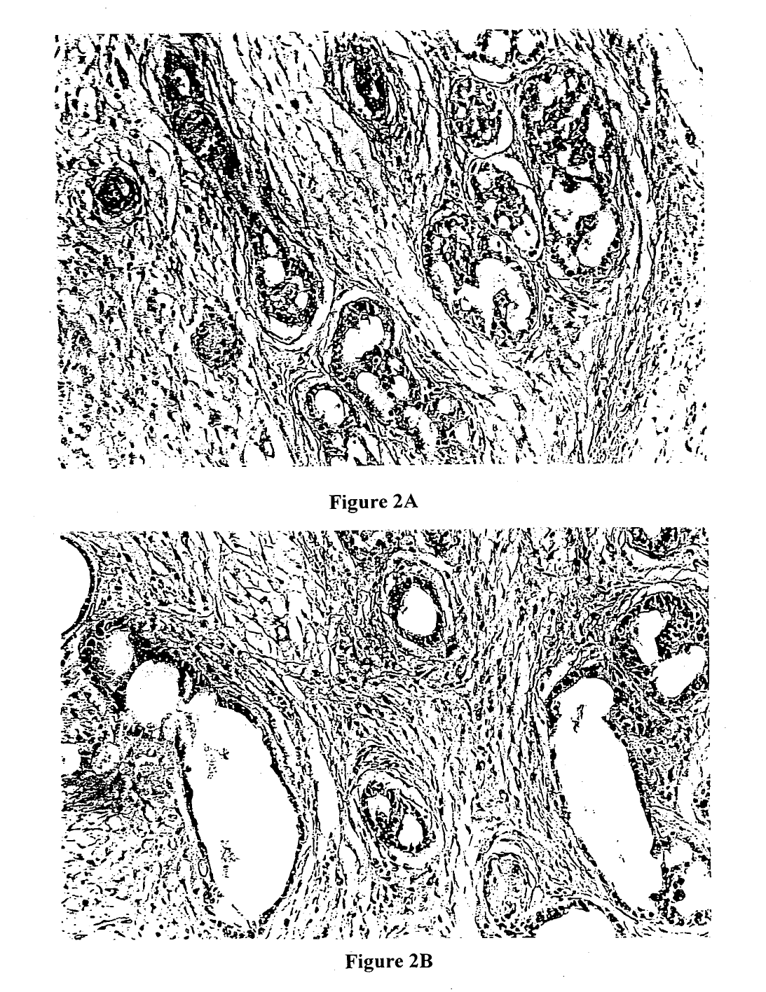

Similar regularities were found in pancreatic transplants. There was preservation of the structure of the acinar epithelium in the pancreatic transplant group that received irradiation as shown in FIG. 2A, whereas the non-irradiated group exhibited atrophy of the acinar epithelium as shown in FIG. 2B.

As shown below in Table 1, the number of blood vessels in the irradiated rabbit kidney and pancreas transplants in nude mice was more than without irradiation. In addition, the size of the vessels was bigger in irradiated transplants.

TABLE 1

| | Number | Size of | Number of |
|---|---|---|---|
| Group | of Mice | Transplant, mm² | Vessles |
| Renal, no irradiation | 12 | 34.8 ± 7.2 | 9.8 ± 1.02 |
| Renal, with irradiation | 12 | 52.0 ± 7.5 | 44.8 ± 4.25 |
| Pancreas, no irradiation | 6 | 15.3 ± 1.6 | 14.2 ± 1.28 |
| Pancreas, with irradiation | 6 | 27.3 ± 2.9 | 23.7 ± 2.02 |

The example set forth above is provided to give those of ordinary skill in the art with a complete disclosure and description of how to make and use the preferred embodiments of the compositions, and is not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

We claim:

1. A method of transplanting tissue into a recipient to enhance vascularization of the tissue comprising the steps of:
(a) preparing the tissue for transplantation into a transplantation site on or in the recipient;
(b) implanting the tissue into a transplantation site in the recipient; and
(c) applying low-power laser to the tissue.

2. The method of claim 1, wherein the tissue is from natural sources.

3. The method of claim 1, wherein the tissue is a tissue-engineered construct.

4. The method of claim 1, wherein the tissue is autologous.

5. The method of claim 1, wherein the tissue is allogenic.

6. The method of claim 1, wherein the tissue is heterogenic.

7. The method of claim 1 which further comprises treating said tissue with recipient endothelial cells.

8. A method for treating a human recipient with diabetes comprising transplanting an allogenic islet into a transplantation site of the recipient and administering low-power laser to the islet.

9. The method of claim 8 which further comprises treating said tissue with recipient endothelial cells.
